SEQUENCE LISTING (67 sequences)

SEQ ID NO: 1 - protein, 15 aa, Hepatitis C Virus
QLINTNGSWHINSTA

SEQ ID NO: 2 - protein, 13 aa, Hepatitis C Virus
NTGWLAGLFYQHK

SEQ ID NO: 3 - protein, 20 aa, Hepatitis C Virus
LNCNESLNTGWLAGLFYQHK

SEQ ID NO: 4 - protein, 9 aa, Hepatitis C Virus
RGRQPIPKA

SEQ ID NO: 5 - protein, 12 aa, Hepatitis C Virus
CVVIVGRIVLSG

SEQ ID NO: 6 - protein, 16 aa, Hepatitis C Virus
PYCWHYPPKPCGIVPA

SEQ ID NO: 7 - protein, 15 aa, Hepatitis C Virus
GNHVSPTHYVPESDK

SEQ ID NO: 8 - protein, 36 aa, Hepatitis C Virus
QLINTNGSWHINSTALNCNESLNTGWLAGLFYQHKF

SEQ ID NO: 9 - protein, 20 aa, Hepatitis C Virus
LNCNESLNTGWLAGLFYQHK

SEQ ID NO: 10 - protein, 14 aa, Hepatitis C Virus
WHINSTALNCNESL

SEQ ID NO: 11 - protein, 9 aa, Influenza virus
LSLRNPILV

SEQ ID NO: 12 - protein, 10 aa, Influenza virus
GLFGAIAGFC

SEQ ID NO: 13 - protein, 9 aa, Influenza virus
SLLTEVETC

SEQ ID NO: 14 - protein, 27 aa, Influenza virus
FDERRNKYLEEHPSAGKDPKKTGGPIC

SEQ ID NO: 15 - protein, 16 aa, Influenza virus
NPGNAEIEDLIFLARC

SEQ ID NO: 16 - protein, 10 aa, Influenza virus
TYQRTRALVC

SEQ ID NO: 17 - protein, 19 aa, Influenza virus
DAVATTHSWIPKRNRSILC

SEQ ID NO: 18 - protein, 11 aa, Influenza virus
GILGFVFTLTV

SEQ ID NO: 19 - protein, 10 aa, Influenza virus
SWPDGAELPF

SEQ ID NO: 20 - protein, 11 aa, Influenza virus
RRSGAAGAAVK

SEQ ID NO: 21 - protein, 11 aa, Influenza virus
RRSGAAGAAVK

SEQ ID NO: 22 - protein, 11 aa, Influenza virus
QLVWMACHSAA

SEQ ID NO: 23 - protein, 8 aa, Influenza virus
EVETPIRN

SEQ ID NO: 24 - protein, 15 aa, Influenza virus
MSLLTEVETIPRNEW

SEQ ID NO: 25 - protein, 7 aa, Influenza virus
KRGPGSG

SEQ ID NO: 26 - protein, 10 aa, Influenza virus
TNQEQTSLYV

SEQ ID NO: 27 - protein, 14 aa, Influenza virus
EGSYPKLKNSYVNK

SEQ ID NO: 28 - protein, 10 aa, Influenza virus
YPYDVPDYAS

SEQ ID NO: 29 - protein, 12 aa, Influenza virus
GFFSRLNWLTKS

SEQ ID NO: 30 - protein, 7 aa, Influenza virus
RSQQTII

SEQ ID NO: 31 - protein, 25 aa, Influenza virus
VTGLRNIPSIQSRGLFGAIAGFIEG

SEQ ID NO: 32 - protein, 11 aa, Influenza virus
SFERFEIFPKE

SEQ ID NO: 33 - protein, 9 aa, Influenza virus
YPYDVPDYA

SEQ ID NO: 34 - protein, 10 aa, Influenza virus
WLTEKEGSYP

SEQ ID NO: 35 - protein, 24 aa, Influenza virus
CPKYVKQNTLKLATGMRNVPEKQT

SEQ ID NO: 36 - protein, 24 aa, Influenza virus
GVHHPSTNQEQTSLYVQASGRVTV

SEQ ID NO: 37 - protein, 7 aa, Influenza virus
NVPEKQT

SEQ ID NO: 38 - protein, 13 aa, Influenza virus
VTGLRNIPSIQSR

SEQ ID NO: 39 - protein, 39 aa, Influenza virus
AMEQMAGSSEQAAEAMEVASQARQMVQAMRTIGTHPSSS

SEQ ID NO: 40 - protein, 20 aa, Influenza virus
GKICNNPHRILDGIDCTLID

SEQ ID NO: 41 - protein, 13 aa, Influenza virus
LTEVETPIRNEWG

SEQ ID NO: 42 - protein, 9 aa, Influenza virus
TYQRTRALV

SEQ ID NO: 43 - protein, 13 aa, Influenza virus
VTGLRNIPSIQSR

SEQ ID NO: 44 - protein, 18 aa, Influenza virus
ALNGNGDPNNMDKAVKLY

SEQ ID NO: 45 - protein, 17 aa, Influenza virus
GTHPSSSAGLKNDLLEN

SEQ ID NO: 46 - protein, 14 aa, Influenza virus
GTLVKTITDDQIEV

SEQ ID NO: 47 - protein, 11 aa, Influenza virus
GFTWTGVTQNG

SEQ ID NO: 48 - protein, 15 aa, Influenza virus
ACKRGPGSGFFSRLN

SEQ ID NO: 49 - protein, 14 aa, Influenza virus
LTKSGSTYPVLNVT

SEQ ID NO: 50 - protein, 17 aa, Influenza virus
HHPSTNQEQTSLYVQAS

SEQ ID NO: 51 - protein, 16 aa, Influenza virus
ITYGACPKYVKQNTLK

SEQ ID NO: 52 - protein, 17 aa, Influenza virus
APIDTCISECITPNGSI

SEQ ID NO: 53 - protein, 15 aa, Influenza virus
ACKRGPGSGFFSRLN

SEQ ID NO: 54 - protein, 5 aa, Influenza virus
LKLAT

SEQ ID NO: 55 - protein, 8 aa, Influenza virus
RNVPEKQT

SEQ ID NO: 56 - protein, 8 aa, Influenza virus
PKYVKQNT

SEQ ID NO: 57 - protein, 7 aa, Influenza virus
NVPEKQT

SEQ ID NO: 58 - protein, 24 aa, Influenza virus
MSLLTEVETPIRNEWGCRCNGSSD

SEQ ID NO: 59 - protein, 18 aa, Human immunodeficiency virus
GIRPVVSTQLLLNGSLAE

SEQ ID NO: 60 - protein, 20 aa, Human immunodeficiency virus
NTRKSIRIQRGPGRAFVTIG

SEQ ID NO: 61 - protein, 25 aa, Human immunodeficiency virus
LPTPRGPNRPEGIEEEGGERDRDRS

SEQ ID NO: 62 - protein, 27 aa, Human immunodeficiency virus
RKRIHIGPGRAFGPKEPFRDYVDRFYK

SEQ ID NO: 63 - protein, 27 aa, Human immunodeficiency virus
GPKEPFRDYVDRFYKRKRIHIGPGRAF

SEQ ID NO: 64 - protein, 32 aa, Human immunodeficiency virus
RKRIHIGPGRAFYTTKNGPKEPFRDYVDRFYK

SEQ ID NO: 65 - protein, 32 aa, Human immunodeficiency virus
GPKEPFRDYVDRFYKRKRIHIGPGRAFYTTKN

SEQ ID NO: 66 - protein, 34 aa, Human immunodeficiency virus
NKRKRIHIGPGRAFYTTKNGPKEPFRDYVDRFYK

SEQ ID NO: 67 - protein, 34 aa, Human immunodeficiency virus
GPKEPFRDYVDRFYKNKRKRIHIGPGRAFYTTKN

(19) United States
(12) Patent Application Publication
(10) Pub. No.: US 2010/0322943 A1
(43) Pub. Date: Dec. 23, 2010

(54) THERAPEUTIC AND DIAGNOSTIC AFFINITY PURIFIED SPECIFIC POLYCLONAL ANTIBODIES

(76) Inventor: Thomas Cantor, Santee, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/816,520

(22) Filed: Jun. 16, 2010

Related U.S. Application Data
(60) Provisional application No. 61/187,984, filed on Jun. 17, 2009.

(51) Int. Cl. A61K 39/42; A61P 31/12; A61P 31/10; A61P 33/00

(52) U.S. Cl. 424/147.1; 424/159.1

(57) ABSTRACT

Provided herein are compositions that include a mixture of polyclonal antibodies obtained from the plasma of a plurality of individuals, and methods of making and using the same.

### CROSS-REFERENCE TO RELATED APPLICATIONS

This application claims priority to U.S. Provisional Patent Application No. Application No. 61/187,984, filed Jun. 17, 2009, to Thomas Cantor, and entitled “THERAPEUTIC AND DIAGNOSTIC PURIFIED SPECIFIC POLYCLONAL ANTIBODIES,” the contents of which are hereby incorporated by reference in its entirety.

### REFERENCE TO SEQUENCE LISTING, TABLE, OR COMPUTER PROGRAM LISTING

The present application is being filed along with a sequence listing in electronic format. The sequence listing is provided as file entitled SCNTLB.001A.txt, created Jun. 15, 2010, which is 13.6 KB in size. The information in the electronic format of the sequence listing is incorporated herein by reference in its entirety.

### BACKGROUND OF THE INVENTION

Much research effort has been invested in determining methodologies for promoting and augmenting host defense mechanisms, in order to treat, cure and prevent diseases and disorders caused by infection with pathogens, such as viral pathogens, microbial pathogens, fungal pathogens, parasitical pathogens, and the like. Augmenting the host defenses with artificially acquired immunity, such as passive immunization, may, in many clinical settings, be preferable or complementary to the use of antibiotics.

Artificially acquired immunity can be either actively or passively induced. Actively induced artificially acquired immunity is achieved by administration of a vaccine to a subject, which stimulates the subject to mount a primary response against the antigen without causing symptoms of the disease. Passive immunization is a short-term immunization by the injection of antibodies, such as gamma globulin, that are not produced by the recipient's immune cells. Regarding passive immunization, antibodies can be administered as human or animal plasma or serum, as pooled human immunoglobulin for intravenous (IVIG) or intramuscular (IG) use, as high-titer human IVIG or IG from immunized or convalescing donors, and as monoclonal antibodies (MAb). Alternatively, antibodies purified using a protein A or protein G Sepharose chromatographic step, which specifically binds the Fc region of IgG antibodies, can be used to obtain antibodies useful for passive immunization.

Intravenous immunoglobulins (IVIG) are not uniquely specific antibodies against a particular antigen, but are the full complement of antibodies found in plasma. IVIG have become an important treatment regime for bacterial and viral infections associated with primary and secondary immunodeficiency states. For example, Buckley et al., New Eng. J. Med. 325:110-117 (1991) have reported using IVIG in the treatment of immunodeficiency diseases, and Commetta et al., New Eng. J. Med. 327:234-239 (1992) have described the prophylactic intravenous administration of standard immune globulin and core-lipopolysaccharide immune globulin in patients at high risk of post-surgical infection. IVIG is prepared from the pooled plasmas of large numbers of donors, and tends to have a broad representation of antibodies. Pooled polyvalent human globulins usually contain antibodies for ubiquitous pathogens such as H. influenza type B, pneumococci, Staphylococci, Diphtheria, tetanus, respiratory syncytial virus (RSV), measles, cytomegalovirus (CMV), varicella zoster virus, etc.

IVIG therapy has limitations and drawbacks. Passive immunization depends on the presence of high and consistent titers of antibodies to the respective pathogens in each preparation. For example, antibody concentrations vary from lot-to-lot and between manufacturers. Thus, while intravenous passive immunization has been successful for certain diseases, it has had inconsistent performance against many types of infections.

Monoclonal antibody (MAb) therapy can be used for passive immunization, however, this type of therapy also has many shortcomings. As monoclonal antibodies are clonal by nature, they are specific for one epitope. Thus, monoclonal antibodies only “hit” the target (e.g., target pathogen) once. In addition, pathogens contain multiple virulence factors that can causes diseases in the host. Thus, MAb therapy is not desirable in instances where it is advantageous to provide a wide array of antibodies with specificity to different epitopes, capable of simultaneously “hitting” the target with multiple antibodies that each recognize and bind to different epitopes. Furthermore, many of the well-characterized MAb's are derived from murine sources and as such, even when they are “humanized,” induce strong immunogenicity when administered into the human body, and have weak effector functions.

Accordingly, there exists a need for improved compositions for passive immunization, for example, for the treatment, prevention, or cure of diseases or conditions caused by pathogens such as viruses, bacteria, fungi, and parasites.

Currently, there is no universal cure for Hepatitis C virus (HCV). HCV is a blood-borne infectious virus that infects the liver in humans. HCV infections are often initially asymptomatic, but once established, chronic infection can cause inflammation of the liver (chronic hepatitis). This condition can progress to scarring of the liver (fibrosis), and advanced scarring (cirrhosis). In some cases, those with cirrhosis will go on to develop liver failure or other complications of cirrhosis, including liver cancer. HCV can destroy the liver, necessitating a liver transplant. HCV is spread by blood-to-blood contact. No vaccine against hepatitis C is commercially available. An estimated 150-200 million people worldwide are infected with hepatitis C. About 2% of the US population is infected with HCV. The Centers for Disease Control has estimated that the costs associated with HCV infection in the US are $600 million per year. In the US about 10,000 people die from HCV every year. HCV accounts for about half of all liver transplants. Once HCV infection has been diagnosed it is important to stop or control the viral propogation so that permanent liver damage and cancer do not occur. The current mode of treatment for HCV infections is administration of alpha and beta Interferon which promotes the destruction of the virus envelope. This therapy results in only a 30%-40% cure rate.

Currently, there is no treatment or cure for the H1N1 influenza and/or H1N5 influenza.

There is a need for a rapid, natural treatment for HCV that does not have side effects, or has minimal side effects. Further, there is a need for a effective, natural treatment for H1N1 and related influenza strains.

### SUMMARY OF THE INVENTION

Provided herein are improved compositions for passive immunization against pathogenic targets. Specifically, embodiments disclosed herein relate to compositions, as well as methods of making and using the same, that include a pool of polyclonal antibodies derived from the plasma or gamma globulin from more than one individual. The inclusion of several individuals as donors making up the pool used to produce the affinity purified antibodies assures that there will be a composition of antibodies against a sufficient variety of epitopes so as to make this passive immunization effective. The polyclonal antibodies have been processed using affinity separation techniques, to purify, or substantially purify, antibodies specific for the target pathogen (i.e., that specifically bind to target-pathogen derived antigens). Also provided herein are cocktails, that provide a mixture of monoclonal antibodies specific for different epitopes specific for the target pathogen.

Some embodiments provide a composition that includes a mixture of polyclonal antibodies obtained from a plurality of individual subjects and a pharmacologically-acceptable carrier wherein the mixture of polyclonal antibodies includes antibodies that specifically bind to at least one antigen of a target pathogen, and wherein the polyclonal antibodies or the mixture has been processed to substantially separate antibodies that do not specifically bind to the at least one antigen of said target pathogen from the polyclonal antibodies.

Also provided are methods of making the compositions disclosed herein. In some embodiments, the methods include the steps of providing plasma from a plurality of individual subjects; combining the plasma from the plurality of subjects to obtain a plasma mixture; contacting the plasma mixture with at least one antigen of the pathogenic target or related pathogenic target under conditions wherein the polyclonal antibodies within the plasma that specifically bind to the at least one antigen bind to said at least one antigen; separating the polyclonal antibodies from antibodies (and other non specific proteins) that do not specifically bind the at least one antigen of the pathogenic target; and providing the separated antibodies in combination with a pharmacologically-acceptable carrier.

In some embodiments, the method also includes a step of isolating the gamma globulin component from the plasma mixture prior to the contacting step, wherein the gamma globulin component is contacted with the at least one antigen from said pathogenic target or related pathogenic target. In some embodiments, the methods also include the step of exposing bound polyclonal antibodies to conditions wherein the polyclonal antibodies dissociate from the at least one antigen from the pathogenic target following the separating step; and separating the dissociated polyclonal antibodies from the at least one antigen.

In some embodiments, the separated, dissociated polyclonal antibodies can be concentrated. In some embodiments, the methods also include a step of purifying monomeric forms of the polyclonal antibodies from non-monomeric forms of the polyclonal antibodies.

In some embodiments, the methods include a step of treating the plasma or plasma mixture with an amount of a pathogen-inactivating compound sufficient to inactivate any target pathogen in the plasma or plasma mixture. For example, some embodiments include treating the plasma or plasma mixture with a detergent, such as TritonX-100 or the like and/or a solvent, such as a tri-n-butyl phosphate.

In some embodiments, the methods can include the step of treating the plasma or mixture is processed to substantially remove lipids from the composition.

Also provided are methods of treating individuals with the compositions disclosed herein. Specifically, provided herein are method of treating or preventing a disease caused by a pathogenic target, including the step of: identifying a first subject that is infected with said pathogenic target; and administering to the subject a therapeutically effective amount of a composition comprising a mixture of polyclonal antibodies obtained from a plurality of individual subjects other than the first subject, wherein the polyclonal antibodies specifically bind to the pathogenic target, and wherein the polyclonal antibodies have been processed to separate antibodies that do not specifically bind to the pathogenic target from said polyclonal antibodies.

The target pathogen of the embodiments disclosed herein can be a viral particle, a pathogenic microorganism, a pathogenic fungus, or a pathogenic parasite. In some embodiments, the target pathogen is a viral particle, such as an HCV viral particle, an influenza viral particle (e.g, an influenza A viral particle such as H1N1, H1N5, or the like), or the like.

In preferred embodiments, the plurality of individuals from whom the polyclonal antibodies against the target pathogen are derived are human.

In some embodiments, the plurality of individuals from whom the polyclonal antibodies against the target pathogen are derived include individuals that are infected with said pathogenic target. In some embodiments, all of the individuals are infected with the target pathogen. In other embodiments, not all of the individuals are infected with the target pathogen. In some embodiments, none of the individuals are infected with the target pathogen. In some embodiments, all of the individuals have been exposed to the target pathogen, whereas in some embodiments, not all of the individuals have been exposed to the target pathogen.

In some embodiments, the plurality of individuals from whom the polyclonal antibodies are derived are collectively infected with a plurality of strains of the target pathogen. For example, in some embodiments, the plurality of individuals can be collectively infected or exposed to with a plurality of HCV strains. In some embodiments, the plurality of individuals can be collectively infected with and/or exposed to a plurality of H1N1 strains.

In some embodiments, the amount polyclonal antibodies in the compositions disclosed herein can include between about 0.01 to about 1mg polyclonal antibodies, or more, e.g., 10 mg, per milliliter. In some embodiments, the compositions disclosed herein can include pharmaceutically acceptable carriers that are suitable for infusion, or transdermal delivery. For example, in some embodiments, the pharmaceutically acceptable carrier can include maltose and/or polysorbate 80.

### BRIEF DESCRIPTION OF THE DRAWINGS

FIG. 1 shows the calibration curve for the gel filtration column. The standards were thyroglobulin (670 KDa), bovine gamma globulin (158 KDa), chicken ovalbumin (44 KDa), equine myoglobin (17 KDa) and vitamin B12 (1.35 KDa).

FIG. 2 shows the gel filtration results of normal human plasma eluate from an affinity column prepared with H1N1 PB1-F2 (62-70) antigen.

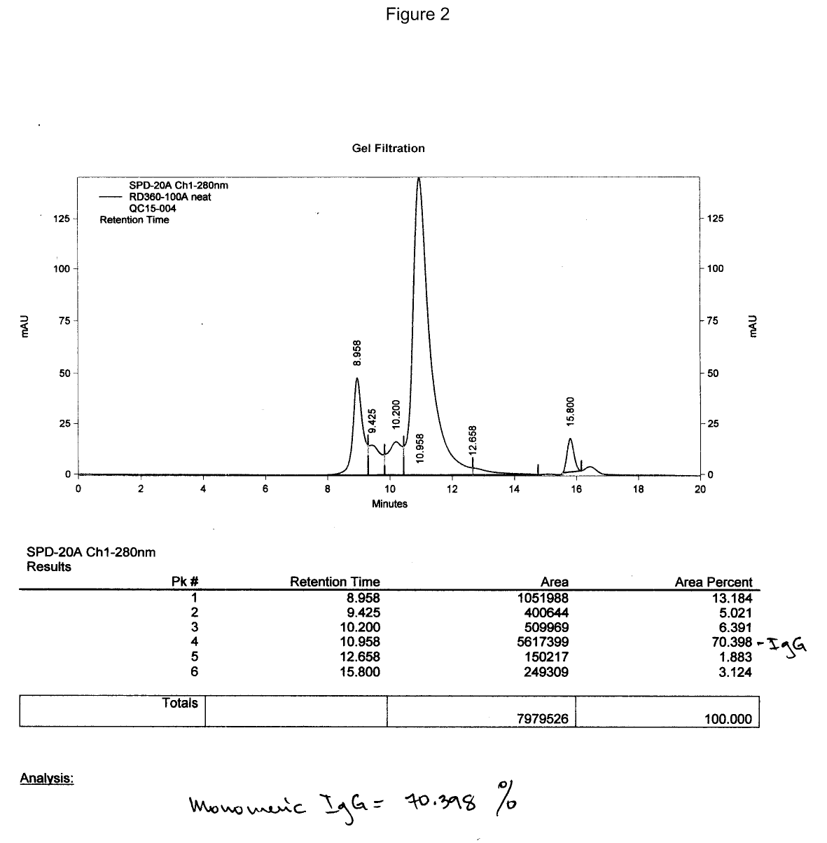

### DETAILED DESCRIPTION OF THE PREFERRED EMBODIMENT

The present disclosure relates to Applicants' discovery of improved compositions for passive immunization against pathogenic targets. The compositions provided herein advantageously enable the delivery of a high concentration of polyclonal, target specific antibodies obtained from a plurality of different individuals. In some embodiments, the therapy can be administered at regular intervals for a period of time, e.g., every two weeks for a 60 week period, where each administration is made up of the targeted antibodies extracted from approximately six liters human plasma. Accordingly, provided herein are compositions, as well as methods of making and using the same, that include a pool of polyclonal antibodies derived from the plasma or gamma globulin from more than one individual. The polyclonal antibodies have been processed (e.g., using affinity separation techniques) to purify, or significantly or substantially purify, antibodies specific for the target pathogen (i.e., that specifically bind to target-pathogen derived antigens), away from non-target-specific antibodies and other plasma or gamma globulin components. As discussed further below, the compositions described herein eliminate the need to administer large volumes of, for example, non specific immunoglobulin, to individuals in order to achieve delivery of an amount of antibody that is target-specific to a subject in need thereof. Furthermore, because the compositions disclosed herein are derived from plasma (or gamma globulin fraction of plasma) from a plurality of individuals, and are polyclonal, the compositions can advantageously include antibodies that recognize numerous epitopes on the target pathogen, as well as various strains or types of the target pathogen.

One advantage of some of the treatment methods disclosed herein is the ability to largely or wholly knock down an infectious pathogen, and to treat or potentially even cure the underlying infection. Another advantage of the treatment method disclosed herein is that, in some embodiments, the long-term treatment involving multiple administrations of the antibodies at regular intervals over a period of time, e.g., over the course of 2 weeks, 4 weeks, 6 weeks, 8 weeks, 10 weeks, 15 weeks, 20 weeks, 25 weeks, 30 weeks, 35 weeks, 40 weeks, 45 weeks, 50 weeks, 55 weeks, 60 weeks, or more, or any amount of time in between, of antibody, assures that virus concealed from antibodies within cells will during that time emerge from cells and be inactivated by the high levels of circulating antibodies. The immune system is a powerful weapon, but in many instances of infection, it is simply overwhelmed or functionally lacking the requisite quantity and/or diversity and specificity of antibodies required to destroy the infection. Passive immunization with a monoclonal antibody can provide some degree of neutralization of he target pathogen or epitope, but due to the single-epitope nature of the monoclonal binding, the results are often sub-optimal. Similarly, polyclonal antibodies from a single individual may prove to be inadequate both in terms of diversity of specificity and quantities. However, pooled antibody from a large number of individuals as in the embodiments described herein, can provide a large number of antibodies that bind to a larger number of epitopes on each virus or other pathogen, and can thus dramatically neutralize and/or block pathogen particle counts in a manner not attainable with other passive monoclonal immunotherapy protocols. In some instances, this can reduce pathogen load to a point where any remaining infection is controlled or even eliminated by the patient's own immune system.

It is intended that where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the embodiments, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein may also be used in the practice or testing of the embodiments, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

Some embodiments disclosed herein relate to compositions comprising a mixture, or pool, of polyclonal antibodies obtained from a plurality of different subjects.

As used herein, the term “polyclonal antibodies” refers to a heterogeneous pool of antibodies produced by a number of different B lymphocytes. Different antibodies in the pool recognize and specifically bind different epitopes. The term “epitope” as used herein refers to a polypeptide sequence of at least about 3 to 5, preferably about 5 to 10 or 15, and not more than about 1,000 amino acids (or any integer there between), which define a sequence that by itself or as part of a larger sequence, binds to an antibody generated in response to such sequence. A target antigen may contain linear and/or discontinuous epitopes. There is no critical upper limit to the length of the fragment, which may (for example) comprise nearly the full-length of the antigen sequence, or even a fusion protein comprising two or more epitopes from the target antigen. An epitope for use in the subject invention is not limited to a polypeptide having the exact sequence of the portion of the parent protein from which it is derived. Indeed, some viral genomes are conserved. However, some viral genomes are in a state of frequent mutation from episode to episode, and contain several variable domains which exhibit relatively high degrees of variability between isolates. Thus the term “epitope” encompasses sequences identical to the native sequence, as well as mutations or modifications to the native sequence, such as deletions, additions and substitutions (generally conservative in nature).

The term “monoclonal antibody” as used herein refers to an antibody obtained from a population of substantially homogeneous antibodies, i.e., the individual antibodies comprising the population are identical except for possible naturally-occurring mutations that may be present in minor amounts.

As used herein, reference to an antibody that “specifically binds” or “selectively binds” to an epitope of an antigen of a target pathogen, refers to an antibody that does not bind other unrelated antigens, or with substantially reduced affinities. By way of example, anti-HCV antibodies can bind HCV and show no binding above about 1.5 times background for HBV antigens, or antigens from other unrelated target pathogens. Likewise, anti-H1N1 antibodies show no binding above 1.5 times background to antigens from unrelated pathogens. In some embodiments, the cross-reactivity of an antibody that “specifically binds” an antigen with an unrelated antigen, is less than about 35%, 33%, 30%, 27%, 25%, 20%, 15%, or 10%, as measured by routine methods, e.g., by competition ELISA or by measurement of K_{d} with BIACORE™ (GE Healthcare Life Sciences, Piscataway, N.J.), or KINEXA™ (Sapidyne Instruments, Inc., Boise, Id.) assay.

As used herein, the term “related pathogen” refers to a pathogen that comprises antigens that can be recognized and specifically bound and/or neutralized by the specific antibodies that are isolated with the target pathogen. By way of example, some antigens present in H1N5 influenza virus can be used to isolate or purify antibodies that specifically bind to, and/or neutralize H1N1 influenza virus.

The mixture of polyclonal antibodies includes polyclonal antibodies from a plurality of different subjects. In some contexts, the terms “individual,” “host,” “subject,” and “patient” are used interchangeably to refer to an animal. “Animal” includes vertebrates and invertebrates, such as fish, shellfish, reptiles, birds, and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

In some embodiments, the mixture of polyclonal antibodies can be obtained from 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 55, 60, 65, 70, 75, 80, 85, 90, 95, 100, or more, individual subjects, or any number in between.

In some embodiments, all of the individual subjects from whom the pool of polyclonal antibodies are obtained are infected with the target pathogenic organism.

In other embodiments, some, but not all of the subjects from whom the pool of polyclonal antibodies are obtained are infected with the target pathogen. In some embodiments, none of the individuals show symptoms or clinical indications of being infected with the target pathogen. In some embodiments, some or all of the individuals have been exposed to the target pathogenic organism, but do not show the symptoms or clinical indications of being infected with the target pathogenic organism. As used herein, an individual “infected with” a target pathogen refers to individuals in which the target pathogen is present. As used herein, an individual that has been “exposed to” a target pathogen refers to an individual that was at one point in time infected with a target pathogen, but in whom the target pathogen is not necessarily still present. As discussed further below, routine diagnostic tests can be used to determine whether an individual is infected with, or has been exposed to, a target pathogen. Preferably, all or almost all of the individuals from whom the polyclonal antibodies are obtained have mounted an immune response against the target pathogen, and, as such, have plasma that contains a detectable concentration of target-specific antibodies. In some embodiments, the individuals from whom the polyclonal antibodies are obtained can be individuals immunized against the pathogen or related pathogen.

It has been reported that a pool of immunoglobulin or plasma from several individuals that did not test positive for HCV offers significant protection against HCV infection. See, Piazza et al. (1997) Arch. Intern. Med. 157(14):1537-1544. Likewise, Applicants have discovered that normal human plasma contains antibodies that bind to antigens derived from H1N5/avian flu. Accordingly, in some embodiments, none of the individuals have been infected or exposed to the target pathogen.

The compositions described herein include antibodies that specifically bind to at least one antigen of a target pathogen. The target pathogen can be, for example, an intracellular parasite, an extracellular parasite (such as a bacterium, a protozoa, and a helminth, for example those which cause leprosy, tuberculosis, leishmania, malaria, or schistosomiasis) or a virus.

Representative examples of viral pathogens include, without limitation, HIV, Hepatitis A, Hepatitis B, Hepatitis C, rabies virus, Herpes viruses, Cytomegalovirus, poliovirus, influenza virus, meningitis virus, measles virus, mumps virus, rubella, varicella, pertussis, encephalitis virus, papilloma virus, yellow fever virus, Influenza virus A (e.g., H1N1 influenza virus, H2N2 influenza virus, H1N5 influenza virus, H2N2 influenza virus, H5N1 influenza virus, H7N7 influenza virus, H1N2 influenza virus, H9N2 influenza virus, H7N2 influenza virus, H7N3 influenza virus, H1ON7 influenza virus), Influenza virus B, Influenza virus C, Epstein-Barr virus, respiratory syncytial virus, parvovirus, chikungunya virus, haemorrhagic fever viruses, Klebsiella, a virus of the paramyxoviridae family, including human paramyxoviridae viruses such as paramyxoviruses (e.g. parainfluenza virus 1, parainfluenza virus 2, parainfluenza virus 3 and parainfluenza virus 4), morbilliviruses (e.g. measles virus) and pneumoviruses (e.g., respiratory syncytial virus); a non-human paramyxoviridae virus, such as canine distemper virus, bovine respiratory syncytial virus, Newcastle disease virus and rhinderpest virus.

Additional examples of pathogenic microorganisms include gram negative bacteria, including but not limited to, Escherichia coli, Enterobacter aerogenes, Enterobacter cloacae, Klebsiella pneumoniae, Proteus mirabilis, Proteus vulgaris, Morganella morganii, Providencia stuartii, Serratia marcescens, Citrobacter freundii, Salmonella typhi, Salmonella paratyphi, Salmonella typhi murium, Salmonella virchow, Shigella spp., Yersinia enterocolitica, Acinetobacter calcoaceticus, Acinetobacter baumannii, Flavobacterium spp., Haemophilus influenzae, Pseudomonas aeruginosa, Campylobacter jejuni, Vibrio parahaemolyticus, Brucella spp., Neisseria meningitidis, Neisseria gonorrhoea, Bacteroides fragilis, and Fusobacterium spp.

Other examples of target pathogens include pathogenic microorganisms such as Gram-positive bacteria, including but not limited to, Streptococcus pyogenes (Group A), Streptococcus pneumoniae, Streptococcus GpB, Streptococcus viridans, Streptococcus GpD-(Enterococcus), e.g. Enterococcus faecium, Enterococcus faecalis, Streptococcus GpC and GpG, Staphylococcus aureus, Staphylococcus epidermidis, Bacillus subtilis, Bacillus anthraxis, Listeria monocytogenes, Anaerobic cocci, Clostridium spp., and Actinomyces spp.

Still other target pathogens include extracellular parasite, such as a protozoan (such as babesia), and a helminth, including extracellular parasites which cause leprosy, tuberculosis, leishmania, malaria, or schistosomiasis and the like.

Because the compositions of some embodiments described herein are derived from polyclonal antibodies from a plurality of individuals, the compositions described herein advantageously include antibodies that can recognize different epitopes on an antigen or large number of antigens on the target pathogen. Furthermore, as discussed below, in some embodiments, the individuals from whom the polyclonal antibodies are obtained can be collectively infected with, and therefore have plasma that contains antibodies specific for several different strains or types of the target pathogen. For example, in some embodiments, the individuals from whom the polyclonal antibodies are obtained are, collectively, infected with several strains of the same species of target pathogen. For example, in some embodiments, the individuals collectively are infected with various strains of HCV. For example, the individuals collectively can be infected with one or more HCV genotypes of groups 1, 2, 3, 4, 5, 6, 7, 8, 9, 10 and 11. In some embodiments, the individuals can collectively be infected with one or more subgroups (e.g., 1a, 1b, 2a, 2b, 2c, 3a, 4a, 4b, 4c, 5a, 6a, 7a, 7b, 8a, 8b, 9a, 10a, 11a, or the like) of HCV of a particular HCV genotype(s). As such, in some embodiments, the compositions disclosed herein in which the target pathogen is, for example, HCV, include polyclonal antibodies specific for, and effective against, several different types of HCV. Furthermore, due to the polyclonal nature of the compositions, the antibodies can simultaneously recognize and bind to many epitopes on the target pathogen, such as HCV.

In some embodiments, the individuals can be infected with various strains of H1N1 influenza virus (swine flu), or H5N1 influenza virus (avian flu).

One of the limitations of currently available methods of passive immunization, e.g., IVIG, is that the injection of large amounts of antibodies in individuals has side effects. For example, IVIG products have been reported to be associated with renal dysfunction, acute renal failure, osmotic nephrosis and death. Accordingly, it is recommended that IVIG products are administered at the minimum concentration available and the minimum rate of infusion practicable. The number of target-specific antibodies present in IVIG is relatively low compared to the total amount of immunoglobulins. Due to the limitations on the volume or concentration of antibodies that can be safely administered to a subject, IVIG recipients end up receiving only a limited quantity of target-specific antibodies. Applicants have discovered that by separating away antibodies that are specific for antigens of the target pathogen from non-specific antibodies, it is possible to generate an improved composition for passive immunization that has a high concentration of target-specific antibodies. In some embodiments, the polyclonal antibodies have been processed to substantially separate antibodies that do not specifically bind to an antigen from the target pathogen from antibodies that specifically bind to an antigen from the target pathogen. As used herein, the term “substantially separated” is intended to mean, e.g., 30, 40, 50, 60, 70, 80, 90, 95, 96, 97, 98, 99, 100% free of antibodies that are not specific for antigens of the target pathogen. Accordingly, the recipients of the compositions disclosed herein can receive a greater amount of target-specific antibodies in each administration.

Because the polyclonal antibodies of the mixtures described herein have been substantially separated and/or substantially purified away from antibodies that are not specific for the target pathogen, the compositions provided herein can advantageously provide a higher concentration of target-specific antibodies, when compared, for example, to the concentration of target-specific antibodies in other therapeutic passive immunizations, such as IVIG and the like, derived from non-processed gamma globulin from multiple individuals. For example, in some embodiments, greater than 60%, 65%, 70%, 75%, 80%, 85%, 86%, 87%, 88%, 89%, 90%, 91%, 92%, 93%, 94%, 95%, 96%, 97%, 98%, or 99%, or any fraction in between, of the antibodies in the compositions described herein are specific for an antigen of the target pathogen.

Target-specific antibodies can be separated and/or purified away from non-specific antibodies using conventional techniques. For example, in some embodiments, affinity chromatography is used to purify the target-specific antibodies away from non-specific antibodies and other proteins present in plasma. Preferably, one or more antigens derived from the target pathogen are used in the affinity purification of the target-specific antibodies from the non-specific antibodies. By way of example, in some embodiments, one or more antigens or non-infectious antigen components from the target pathogen can be immobilized onto a solid support, e.g., either covalently or non-covalently. By way of example, in some embodiments one or more target-derived antigens is coupled to a column matrix, such as Sepharose, or the like. The matrix bound by the target-specific antigens can be used to generate an affinity column. The skilled artisan will appreciate, however that any appropriate technique can be used to separate target-specific antibodies away from non-specific antibodies and contaminants, such as clotting factors, hormones, serum albumin and the like.

### Preparation of Compositions for Passive Immunization

In accordance with the embodiments disclosed herein, provided herein are methods of preparing the disclosed compositions. In some embodiments, the methods involve obtaining plasma from a plurality of individual subjects. As described above, in some embodiments, one or more of the subjects is infected with (e.g., tests positive for) the target pathogen. In other embodiments, some or all of the subjects have not tested positive for the target pathogen, for example, using routine diagnostic methods, such as testing the subject's plasma for the presence of the target pathogen and/or antibodies against the target pathogen. In some embodiments, the individuals have been exposed to the target pathogen. In some embodiments, not all of the individuals have been exposed to the target pathogen.

Plasma from the plurality of subjects can be processed to separate and/or purify the target-specific antibodies away from the non-specific antibodies and contaminants in the plasma. In some embodiments, the plasma is processed to remove the non-specific antibodies and contaminants, and then combined to form a mixture. In other embodiments, the plasma from the plurality of individuals is pooled to form a plasma mixture. The plasma mixture is subsequently processed to remove the non-specific antibodies and contaminants.

In some embodiments, the plasma is processed to isolate the gamma globulin fraction. “Gamma globulin” refers to a class of the serum proteins with a defined electrophoretic mobility. IgG proteins are the major component of the gamma globulin fraction. In some embodiments, gamma globulins are isolated from plasma before pooling the plasma from the plurality of individuals. Alternatively, the plasma from the plurality of individuals can be pooled, and the gamma globulin fraction can be isolated from the pooled plasma. Isolation of the gamma globulin fraction can be achieved by any method known to those skilled in the art. See, e.g., U.S. Patent Application Publication No. 2008/0242844; See also U.S. Pat. Nos. 6,069,236, 4,719,290, 4,482,483, and 5,177,194. The gamma globulin fraction can then be further processed in order to separate and/or purify target-specific antibodies away from non-specific antibodies using routine methods. Exemplary, non limiting, purification/separation techniques useful in the embodiments disclosed herein are discussed below.

In some embodiments, plasma or gamma globulin fractions can be partially purified prior to processing the plasma or gamma globulin to remove non-specific antibodies. By way of example, in some embodiments, lipids and/or hormones are removed from the plasma/gamma globulin samples prior to affinity purification, e.g., treatment with organic solvents such as cholroform or ether, adsorption techniques that use fumed Silica as CAB-O-SIL™ (Cabot Corp., Boston, Mass.), Protein A purification, ammonium sulfate fractionation, purification with caprylic acid, freeze/thawing, charcoal adsorption or the like. See, e.g., Handbook of Therapeutic Antibodies, Dubel, S. (Ed.), Wiley, Hoboken, N.J. ©2007; Therapeutic Antibodies: Methods and Protocols, Dimitrov, A (Ed.), Humana Press, New York, N.Y., ©2009.

In some embodiments, the plasma or gamma globulin fractions can be treated to eliminate, or substantially eliminate any target pathogens that are present in the plasma or gamma globulin fractions. For example, the plasma and/or gamma globulin fraction can be treated with a pathogen-inactivating compound to inactivate any target pathogen. By way of example, the plasma and/or gamma globulin fractions can be treated with a detergent such as Triton-X100 or the like. In some embodiments, the plasma and/or gamma globulin fractions can be treated with tri-n-butyl phosphate. In some embodiments, the target pathogen in the plasma or gamma globulin fractions can be inactivated by acid treatment. In some embodiments, the plasma and or gamma globulin can be filtered, or subjected to chromatographic procedures to eliminate or substantially eliminate target pathogen from the plasma and/or gamma globulin fraction. Some methods of inactivation of viral target pathogens useful in the methods disclosed herein include, but are not limited to those described in Japanese Patent No. 4,260,877, U.S. Pat. No. 6,569,640, U.S. Pat. No. 6,465,168, and U.S. Pat. No. 7,037,534, and Soluk et al. (2008) Am. J. Therapeutics 15(5):435-443, each of which is herein incorporated by reference in its entirety. The skilled artisan will appreciate that any target pathogen-inactivating compounds can be used in the methods described herein. In some embodiments, the plasma and/or gamma globulin fractions can be assayed to test for the presence and/or elimination of the target pathogen.

In some embodiments, the removal of non-specific antibodies and contaminants from plasma or gamma globulin samples involves immunoaffinity separation/purification. For example, in some embodiments, the plasma or gamma globulin preparations described herein are placed into contact with one or more antigens from the target pathogen under conditions that enable antibodies that are specific for antigens of the target pathogen to bind. For example, in some embodiments, the samples are allowed to bind to the one or more antigens coupled to a solid phase under physiological conditions (e.g. in phosphate buffered saline or the like at pH 7.4). Unbound material (e.g., non-specific antibodies and contaminants and the like) can be separated from the polyclonal antibodies that are bound to the target-specific antigens.

In a preferred embodiment, separation of target-specific antibodies from non-specific antibodies and contaminants involves the use of one or more antigens from the target immobilized on a solid support. Target-specific antibodies selectively bind to the immobilized target-derived antigen(s), and are thereby separated from other components present in the plasma or gamma globulin samples, such as non-specific antibodies and contaminants, such as hormones, lipids, serum albumin and the like. Solid supports suitable for the embodiments disclosed herein can be any material known to those of ordinary skill in the art to which the antigen(s) can be immobilized. For example, the solid support can be a bead or disc, such as agarose polysaccharide, polyacrylamide, glass, fiberglass, latex or a plastic material such as polystyrene or polyvinylchloride. The support may also be a magnetic particle or a fiber optic sensor, such as those disclosed, for example, in U.S. Pat. No. 5,359,681. The antigen(s) can be immobilized on the solid support using a variety of techniques known to those of skill in the art, which are amply described in the patent and scientific literature. In the context of the embodiments described herein, the term “immobilization” refers to both noncovalent association, such as adsorption, and covalent bonding, which may be a direct linkage between the antigen and functional groups on the support or may be a linkage by way of a cross-linking agent, for example cyaogen bromide activated Sepharose 4B (GE Healthcare Life Sciences, Piscataway, N.J.).

Attachment of antigen(s) to a solid support can generally be achieved by first activating the target antigen with a bifunctional reagent that will react with both the support and a functional group, such as a hydroxyl or amino group, on the antigen(s). For example, the binding agent may be covalently attached to supports having an appropriate polymer coating using benzoquinone or by activating the support with aldehyde groups which form covalent bonds with primary amines present on the antigen(s) (See, e.g., Pierce Immunotechnology Catalog and Handbook, ©1991, at A12-A13).

The skilled artisan will readily appreciate that the amount of antigen(s) necessary to bind the specific antibodies will vary depending upon the volume of the plasma or gamma globulin fraction to be processed, and the desired amount of purified target pathogen-specific antibodies. For example, in some embodiments, 0.1 μg, 0.5 μg, 5 μg, 10 μg, 25 μg, 100 μg, 150 μg, 200 μg, 250 μg, 500 μg, 1 mg, 2 mg, 3 mg, 4 mg, 5 mg, 7 mg, 10 mg, 15 mg, 20 mg, 25 mg, 30 mg, 35 mg, 40 mg, 45 mg, 50 mg, 55 mg, 60 mg, 65 mg, 70 mg, 75 mg, 100 mg, 150 mg, 200 mg, 250 mg, 300 mg, 350 mg, 400 mg, 450 mg, 500 mg, 550 mg, 600 mg, 650 mg, 700 mg, 750 mg, 1 g, 2 g, 5g, 10 g, of antigen(s) or more, or any amount in between, can be attached to a support, in order to bind the specific antibodies from the plasma and/or gamma globulin fractions.

In embodiments wherein the target pathogen is HCV, any peptide containing an epitope can be used to generate an affinity column for purification of HCV-specific antibodies. A non-limiting list of exemplary HCV peptides useful in the embodiments described herein is provided in Table 1, below.

| SEQ ID NO: | HCV Peptide Sequence |
|---|---|
| 1 | QLINTNGSWHINSTA |
| 2 | NTGWLAGLFYQHK |
| 3 | LNCNESLNTGWLAGLFYQHK |
| 4 | RGRQPIPKA |
| 5 | CVVIVGRIVLSG |
| 6 | PYCWHYPPKPCGIVPA |
| 7 | GNHVSPTHYVPESDK |
| 8 | QLINTNGSWHINSTALNCNESLNTGWLAGLFYQHKF |
| 9 | LNCNESLNTGWLAGLFYQHK |
| 10 | WHINSTALNCNESL |

Other HCV peptides useful in the embodiments described herein can be found, for example, in U.S. Pat. No. 6,436,375, U.S. Pat. No. 7,091,324, U.S. Pat. No. 7,056,658, U.S. Pat. No. 6,692,907, U.S. Pat. No. 6,514,731, U.S. Pat. No. 6,428,792, U.S. Pat. No. 5,766,845, U.S. Pat. No. 5,970,153, U.S. Patent Application Publication No. 2009130135, U.S. Patent Application Publication No. 20070031446, U.S. Patent Application Publication No. 20070014813, and the like, each of which is herein expressly incorporated by reference in its entirety.

In some embodiments, wherein the target pathogen is influenza virus, any peptide containing an epitope can be used to generate an affinity column for purification of influenza virus-specific antibodies. For example, in some embodiments, the peptide can be a hemagluttinin peptide (HA), a nucleocapside protein (NP), a neuramididase (NA), non structural protein 1 (NS1), non structural protein 2 (NS2), M1 or M2, or the like, from influenza virus, or fragments thereof. In addition, recombinant HCV proteins that contain one or more HCV epitopes described herein can be used fro antibody purification and characterization. A non-limiting list of exemplary influenza virus peptides useful in the embodiments described herein is provided in Table 2, below.

| SEQ ID NO: | Influenza Virus Peptide Sequences |
|---|---|
| 11 | LSLRNPILV |
| 12 | GLFGAIAGFC |
| 13 | SLLTEVETC |
| 14 | FDERRNKYLEEHPSAGKDPKKTGGPIC |
| 15 | NPGNAEIEDLIFLARC |
| 16 | TYQRTRALVC |
| 17 | DAVATTHSWIPKRNRSILC |
| 18 | GILGFVFTLTV |
| 19 | SWPDGAELPF |
| 20 | RRSGAAGAAVK |
| 21 | RRSGAAGAAVK |
| 22 | QLVWMACHSAA |
| 23 | EVETPIRN |
| 24 | MSLLTEVETIPRNEW |
| 25 | KRGPGSG |
| 26 | TNQEQTSLYV |
| 27 | EGSYPKLKNSYVNK |
| 28 | YPYDVPDYAS |
| 29 | GFFSRLNWLTKS |
| 30 | RSQQTII |
| 31 | VTGLRNIPSIQSRGLFGAIAGFIEG |
| 32 | SFERFEIFPKE |
| 33 | YPYDVPDYA |
| 34 | WLTEKEGSYP |
| 35 | CPKYVKQNTLKLATGMRNVPEKQT |
| 36 | GVHHPSTNQEQTSLYVQASGRVTV |
| 37 | NVPEKQT |
| 38 | VTGLRNIPSIQSR |
| 39 | AMEQMAGSSEQAAEAMEVASQARQMVQAMRTIGTHPSSS |
| 40 | GKICNNPHRILDGIDCTLID |
| 41 | LTEVETPIRNEWG |
| 42 | TYQRTRALV |
| 43 | VTGLRNIPSIQSR |
| 44 | ALNGNGDPNNMDKAVKLY |
| 45 | GTHPSSSAGLKNDLLEN |
| 46 | GTLVKTITDDQIEV |
| 47 | GFTWTGVTQNG |
| 48 | ACKRGPGSGFFSRLN |
| 49 | LTKSGSTYPVLNVT |
| 50 | HHPSTNQEQTSLYVQAS |
| 51 | ITYGACPKYVKQNTLK |
| 52 | APIDTCISECITPNGSI |
| 53 | ACKRGPGSGFFSRLN |
| 54 | LKLAT |
| 55 | RNVPEKQT |
| 56 | PKYVKQNT |
| 57 | NVPEKQT |
| 58 | MSLLTEVETPIRNEWGCRCNGSSD |

Non-limiting examples of polypeptides useful in embodiments wherein the target pathogen is HIV include peptides or antigenic fragments thereof derived from the HIV-1 Tat protein, e.g., such as those identified in U.S. Pat. No. 7,008,622, the HIV-1 gag protein, gp160 protein, pol, and the like. For example, useful HIV-1 antigens include GIRPVVSTQLLLNGSLAE (SEQ ID NO:59), NTRKSIRIQRGPGRAFVTIG (SEQ ID NO:60), LPTPRGPNRPEGIEEEGGERDRDRS (SEQ ID NO:61), RKRIHIGPGRAFGPKEPFRDYVDRFYK (SEQ ID NO:62), GPKEPFRDYVDRFYKRKRIHIGPGRAF (SEQ ID NO:63), RKRIHIGPGRAFYTTKNGPKEPFRDYVDRFYK (SEQ ID NO:64), GPKEPFRDYVDRFYKRKRIHIGPGRAFYTTKN (SEQ ID NO: 65) NKRKRIHIGPGRAFYTTKNGPKEPFRDYVDRFYK (SEQ ID NO: 66) and GPKEPFRDYVDRFYKNKRKRIHIGPGRA FYTTKN (SEQ ID NO: 67), or any other HIV-1 epitope-containing peptide now known or discovered in the future.

Non-limiting examples of polypeptides useful in embodiments wherein the target pathogen is methicillin-resistant S. aureus include, for example, polypeptides disclosed in U.S. Patent Application Publication No. 2009/130115, and the like.

The skilled artisan will appreciate that the foregoing lists of peptides containing epitopes is not exhaustive, and that any epitope of pathogenic targets now known and discovered in the future can be used in the methods and compositions disclosed herein.

In some embodiments, the peptides above can include a linker, such a cysteine, glycine, or several glycine residues at the C-terminal or N terminal end, in order to facilitate coupling to a solid phase matrix, or the like.

Although several exemplary peptides are listed herein, the skilled artisan will readily appreciate that a wide variety of peptides containing epitopes from target pathogens can be used in the embodiments described herein. Further, the skilled artisan will appreciate that peptides that comprise or include epitopes derived from strains that are closely related to target pathogens can be used in the methods described herein to purify polyclonal antibodies specific for a target pathogens. For example, sequences derived from H1N5 can be used in the methods described herein, to obtain polyclonal antisera for use in treating or preventing the development of symptoms in individuals infected with or exposed to H1N1 influenza virus.

In some embodiments, the immobilized antigen is then incubated with the plasma or gamma globulin, and the antibodies are allowed to bind to the antigen(s). The sample may be diluted with a suitable diluent, such as phosphate-buffered saline (PBS) prior to incubation. In a preferred embodiment, the contact time is sufficient to achieve a level of binding that is at least about 95% of that achieved at equilibrium between bound and unbound antibodies. Those of ordinary skill in the art will recognize that the time necessary to achieve equilibrium may be readily determined by assaying the level of binding that occurs over a period of time. As a general guideline, at room temperature, an incubation time of about 0.01. 0.05, 0.1, 0.5, 1, 5, 10 15, 20, 25, 30, 35, 40, 45, 50, 55, 60, 65, 70, 75, 80, 85, 100, or 120, minutes is generally sufficient.

In some embodiments, the plasma or gamma globulin is passed through an immunoaffinity column. The flow rate of the plasma or gamma globulin sample through the column can be adjusted to ensure that target-specific antibodies bind to the antigen(s) of the affinity column. For example, in some embodiments, the flow rate can be 50 mL/min, 100 mL/min, 150 mL/min, 200 mL/min, 250 mL/min, 300 mL/min, 350 mL/min, 400 mL/min, 450 mL/min, 500 mL/min, 550 mL/min, 600 mL/min, 700 mL/min, 800 mL/min, 900 mL/min, 1000 mL/min, 1100 mL/min, 1200 mL/min, 1300 mL/min, 1400 mL/min, 1500 mL/min, 1600 mL/min, 1700 mL/min, 1800 mL/min, 1900 mL/min, 2000 mL/min, 2100 mL/min, 2200 mL/min 2300 mL/min, 2400 mL/min, 2500 mL/min, or more, or any rate in between, depending on the antibody affinity, the quantity of the immobilized antigens and the dimensions of the column.

In some embodiments, the bound antibody/antigen complexes (e.g., on the solid support) are further washed to remove excess or residual non-specific antibodies or contaminants. The wash conditions can be adjusted using routine methods to ensure that the desired target pathogen-specific antibodies remain bound to the antigen, while other components of the plasma are removed.

Antibodies that specifically bound to the target antigen(s) can then be subsequently dissociated from the immobilized antigen(s) by, for example, altering the pH and/or the salt concentrations of the solution passing over the solid phase antigens bound to specific antibodies. Conditions that promote the dissociation of antibody/antigen complexes are known to those skilled in the art. (See, e.g., Immunology Methods Manual: The Comprehensive Sourcebook of Techniques, Ivan Lefkovits, ed. ©1996, Academic Press).

In some embodiments, monomeric forms of the antibodies can be separated from polymeric or multimeric forms. Methods for separating monomeric forms from polymeric forms are known to those skilled in the art and include gel filtration, ion exchange chromatography (See, e.g. International Patent Application Publication No. WO 99/004970), and the like.

In some embodiments, the dissociated target-specific antibodies can be concentrated. Concentration of the purified target-specific antibodies can be achieved by any number of conventional methods known to those skilled in the art, including but not limited to column chromatography, semi-permeable membrane ultrafiltration, dialysis, concentration centrifugation and the like.

### Monoclonal Antibody Cocktails

Some embodiments provide compositions and methods that use a cocktail or mixture of a plurality of monoclonal antibodies that are each specific for a different epitope of a target pathogen. For example, in some embodiments, the monoclonal antibody cocktail can include 2, 3, 4, 5, 6, 7, 8, 9, 10, 20, 30, or more different monoclonal antibodies that each specifically bind to a different epitope of the target pathogen. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the polypeptides described herein above, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against PRO. Preferably, the binding specificity of monoclonal antibodies produced by the hybridoma cells is determined by immunoprecipitation or by an in vitro binding assay, such as radioimmunoassay (MA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal, or inv vitro in a bioreactor.

The monoclonal antibodies secreted by the subclones may be isolated or purified from the culture medium or ascites fluid by conventional immunoglobulin purification procedures such as, for example, Protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (See, e.g., U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

In some embodiments, one or more of the monoclonal antibodies of the mixture can be a monovalent antibody. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

In some embodiments, one or more of the monoclonal antibodies of the mixture can be a human or humanized monocolonal antibody. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (See, e.g., Jones et al., Nature, 321:522-525 (1986); Riechmann et al., Nature, 332:323-329 (1988); and Presta, Curr. Op. Struct. Biol., 2:593-596 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

### Pharmaceutical Compositions

The dissociated target-specific antibodies can be formulated for administration. The term “pharmaceutical composition” refers to a mixture of a compound of the invention with other chemical components, such as diluents or carriers. The pharmaceutical composition facilitates administration of the compound to a patient. Multiple techniques of administering a compound exist in the art including, but not limited to, intravenous, oral, intramuscular or subcutaneous injection, aerosol, parenteral, and topical administration. Preferably, the compositions described herein are formulated for intravenous, nasal, or parenteral administration.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound. Other compounds useful as pharmaceutically acceptable carriers useful in the embodiments disclosed herein include maltose and polysorbate 80.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Alternatively, one may administer the composition in a local rather than systemic manner, for example, via direct injection of the composition into the patient.

Pharmaceutical compositions for use in accordance with the embodiments described herein thus may be formulated in conventional manner using one or more physiologically acceptable carriers comprising excipients and auxiliaries which facilitate processing of the active compounds into preparations which can be used pharmaceutically, and/or which are suitable for administration to a subject, including a human. Proper formulation is dependent upon the route of administration chosen. Any of the well-known techniques, carriers, and excipients may be used as suitable and as understood in the art; e.g., in Remington's Pharmaceutical Sciences, above.

For injection, the compositions described herein can be formulated in aqueous solutions or lipid emulsions, preferably in physiologically compatible buffers such as Hanks's solution, Ringer's solution, or physiological saline buffer. Proper selection of pH and osmolality are among the considerations important in an injectable or infusible composition.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate injection suspensions. Optionally, the suspension may also contain suitable stabilizers or agents which increase the suspension or stability of the components, or which allow for the preparation of highly concentrated formulations.

Pharmaceutical formulations comprising the compositions disclosed herein can advantageously be formulated for nasal administration. Any intranasal vehicle or formulation may be used, such as aerosols, drops, gels, swabs, and powders. Aerosol formulations typically comprise a solution or fine suspension of the compounds disclosed herein in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Nasal drops, gels, and swabs are well known and have been used for many different bioactive compositions. Such formulations may include conventional excipients, such as thickening agents, preservatives, salts, sugars, or other compounds to adjust ionic strength, mucolytic agents, and the like. Combinations of the antibody preparation and the delivery device are also contemplated; e.g., combinations with a metered dose inhaler, a dry powder inhaler, an atomizer, a dropper, a dropper bottle with or without squeezable sides, or a swab or other applicator.

Pharmaceutical compositions suitable for use in the embodiments disclosed herein include compositions where the target specific polyclonal antibodies, are contained in an amount effective to achieve its intended purpose. More specifically, a therapeutically effective amount means an amount of compound effective to prevent, alleviate or ameliorate symptoms of disease associated with the target pathogen or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). The single, daily, weekly, biweekly, or monthly dosage regimen for an adult human patient may be, for example, the term “therapeutically effective amount/dose” or “inhibitory amount” is used to indicate an amount of an active compound, or pharmaceutical agent, that elicits a biological or medicinal response. This response may occur in a tissue, system, animal or human and includes alleviation of the symptoms of the disease being treated. As used herein with respect to the compositions described herein, e.g., mixtures of affinity purified polyclonal antibodies, the term “therapeutically effective amount/dose” refers to the amount/dose of the compositions disclosed herein (e.g., mixtures of polyclonal antibodies) or pharmaceutical composition containing the mixture, that is sufficient to produce at least a partial pathogen-neutralizing response upon administration to a subject. The dose may be expressed in terms of weight or units that are related to a biological activity.

In some embodiments, the compositions described herein are formulated to deliver an effective neutralizing titer of target pathogen specific antibodies. The term “effective neutralizing titer” as used herein refers to the amount of antibody which corresponds to the amount present in the serum of animals (human or other animal) that has been shown to be either clinically efficacious (in humans) or to reduce virus at pathogenic load by greater than 75%, greater than 80%, greater than 85%, greater than 86%, greater than 87%, greater than 88%, greater than 89%, greater than 90%, greater than 91%, greater than 92%, greater than 93%, greater than 94%, grater than 95%, greater than 96%, greater than 97%, greater than 98%, greater than 99%, or 100%, or any number in between, in, for example, humans, or other subjects.

### Treatment of Conditions and Diseases Caused by Target Pathogens

Some embodiments disclosed herein relate to a method of treating or curing a subject infected with a target pathogen. For example, some embodiments provide a method for treating a disease caused by a pathogenic target, such as a pathogenic virus, a pathogenic microorganism, a pathogenic fungus, or a pathogenic parasite.

In some contexts, the terms “ameliorating,” “treating,” “treatment,” “therapeutic,” or “therapy” do not necessarily mean total cure or abolition of the disease or condition, such as HCV, influenza virus, or the like. Any reduction of pathogen load or viral load or alleviation of any undesired signs or symptoms of a disease or condition, to any extent, can be considered amelioration, and in some respects a treatment and/or therapy. Furthermore, treatment may include acts that may worsen the patient's overall feeling of well-being or appearance.

Accordingly, in some methods a subject infected with a pathogenic target is identified, for example, using routine clinical diagnostic methods or techniques. For example, a subject can be identified as being infected with a pathogenic virus such as HCV, H1N1, H1N5, HIV or the like by determining whether the subject's plasma contains antibodies specific for HCV, H1N1, H1N5, HIV, or any other target pathogen. Several diagnostic tests useful in the embodiments described herein are commercially available. Exemplary HCV diagnostics include Advanced Quality™ One Step HCV Test (Bionike Inc., San Francisco, Calif.), HCV TRI-DOT®, (J. Mitra & Co., Ltd, New Dehli, India), Serdia® (Fujirebio, Inc., Malvern, Pa.), HCV Spot® (Genlabs Diagnostics Pte, Ltd, Singapore Science Park, Singapore), SeroCard™ HCV (Trinity Biotech, PLC, Wicklow, Ireland)). Exemplary HIV diagnostics include Hexagon HIV® (Human GmbH (Berlin, Germany), Clearview Complete HIV ½ (Inverness Medical Professional Diagnostics, Princeton, N.J.), Signal HIV® (Span Diagnostics, Surat, India), and the like.

Nucleic acid based assays can also be used to identify subjects that are infected with a target pathogen. By way of example, a subject can be identified as being infected with microbial pathogens include Xpert™ MRSA test (Cepheid, Sunnyvale, Calif.), BD GeneOhm StaphSR® assay (BD GeneOhm, Franklin Lakes, N.J.), Xpert.™ C. difficile (Cepheid, Sunnyvale, Calif.) or a test for the nucleic acids specific for HCV or HIV.

In some embodiments, individuals that are exposed to the target pathogen are identified.

Once a subject has been identified as being infected with ore exposed to a pathogenic target, the subject can be administered a therapeutically effective amount of a composition disclosed herein, thereby providing polyclonal antibodies that specifically bind to the pathogenic target.

Preferably, the subject is administered the composition in 30 biweekly intravenous doses spanning over a 60 week period or a single dose, such as a single, intravenous injection. In some embodiments, however, the compositions disclosed herein can be administered in multiple doses. For example, in some embodiments, a subject can receive a second, third, fourth, etc. dose, every 24 hours, 36 hours, 48 hours, 72 hours, week, 14 days, month or longer.

In some embodiments, the presence of the target pathogen is immediately measured following the administration of the compositions disclosed herein. In some embodiments, the presence of the target pathogen is measured after a specified time following the administration of the compositions disclosed herein, e.g., one day, two days, three days, five days, a week, two weeks, or more, or any interval of time in between. The subject can be administered one or more additional doses of the compositions disclosed herein. Dosage amount and interval may be adjusted individually to provide sufficient amounts of the polyclonal antibodies to maintain the modulating effects.

In some embodiments, the compositions described herein can be used as a prophylactic to protect against infection with a target organism. For example, in some embodiments, an individual can be identified that are at risk of becoming infected with, or exposed to, a target pathogen. In some embodiments, individuals that have been exposed to, or potentially exposed to, the target pathogen are identified. The individual can be administered one, or multiple doses of the compositions disclosed herein. In other embodiments an individual may have just become infected with the target pathogen and the composition may be given to stop the infection. In other embodiments the target pathogen may have become by mutation resistant to therapy and the composition may be given.

Having now generally described the invention, the same will become better understood by reference to certain specific examples which are included herein for purposes of illustration only and are not intended to be limiting unless other wise specified. All referenced publications and patents are incorporated, in their entirety by reference herein.

### Examples

The following examples are provided to demonstrate particular situations and settings in which this technology may be applied and are not intended to restrict the scope of the invention and the claims included in this disclosure.

A number of publications and patents have been cited hereinabove. Each of the cited publications and patents are hereby incorporated by reference in their entireties.

The following example describes an exemplary procedure for the preparation of a mixture of affinity purified polyclonal antibodies specific for Hepatitis C virus.

### Example 1

### Plasma Source

Approximately 100-1000 individuals are identified as being HCV positive using routine antibody-based diagnostics, e.g., ELISA or RIBA, for example HCV EIA 2.0 (Abbott Laboratories, Abbott Park, Ill.) or ORTHO HCV Version 3.0 ELISA (Ortho-Clinical Diagnostics, Inc. Raritan, N.J.). Approximately 750 mL of plasma is collected from each individual, to yield approximately 750 liters of total HCV-positive plasma. The plasma from the HCV-positive individuals is combined.

### Affinity Column Preparation

One or more synthetic HCV peptides (See, e.g., Table 1) are synthesized using solid-phase synthesis (See, Atherton, E.; Sheppard, R. C. (1989). Solid Phase peptide synthesis: a practical approach. Oxford, England: IRL Press). The synthetic peptides contain epitopes located in diagnostically relevant antigenic regions derived from the E2/NS4/Core regions of HCV.

To prepare the affinity column, an amount of CNBr activated Sepharose 4B Sepharose gel (GE Healthcare Bio-Sciences Corp., Piscataway, N.J.) is measured out in a biological safety cabinet. Approximately 0.5-1.0 mg synthetic HCV eptiope is used per 1 mL Sepharose slurry. One gram dry powder provides approximately 3.0-3.5 ml of gel slurry. The dry powder is suspended in 10 volumes of pre-chilled, 0.2 micron filtered 70% ethyl alcohol made in 0.001 M HCl, pH 3. The gel suspension is mixed on an orbital shaker at 75 rpm for 60 minutes at room temperature in order to sanitize the gel.

The column housing is pre-sanitized with 70% ethyl alcohol, made in 0.001 M HCl, pH 3, for 30 minutes at room temperature.

The HCV peptide is reconstituted in 2-3 volumes of the de-ionized water adjusted to pH 3 with HCl. The reconstituted peptide is diluted into Borate Buffer (0.5 M Borate, 0.5M NaCl, pH 8.5). The final volume of the peptide solution made in the Borate Buffer is approximately 1.0-1.5 volumes of the gel. The peptide solution is filtered through a 0.2 μM sterile filter. The filter is rinsed with a small volume (<0.5 volume of the gel) of the Borate Buffer and the rinse is pooled into the filtered peptide solution.

The sanitized CNBr-activated Sepharose 4B is transferred into autoclaved Buchner sintered glass funnel (funnel method) or pre-sanitized column housing (column method for small test column). A total of 15-20 column volumes (CV) of the 0.2 μM-filtered 0.001 M HCl, pH 3, made in Milli Q water, is applied to wash the gel with periodically stirring or mixing the gel. The flow rate of the pH 3 water is approximately 5 cm/min. The gel is allowed to drain.

0.25 volume of Borate Buffer (005 M Borate, 0.5 M NaCl, pH 8.5), is add into the Buchner funnel or pumped over the column at approximately 5-10 cm/min and the gel is quickly drained.

The gel cake is transferred into the peptide solution stored in a container (funnel method) or into the gel packed in the column housing. The peptide-gel mixture is shaken in a orbital shaker at approximately 75 rpm or rotated end over end at approximately 15 rpm at room temperature for 4 hour and over night at 2-8° C.

The gel is drained and the coupling efficacy is determined by measuring the uncoupled peptide in the spent.

Any remaining active sites on the column are blocked with 2-5 volumes of 1.0 M glycine made in 0.05 M Borate Buffer containing 0.5 M NaCl, pH 8.5, at room temperature for one hour on a shaker at approximately 75 rpm, or a rotator at approximately 15 rpm.

The gel is washed in the Buchner funnel or the column housing at room temperature alternatively with 2-3 volumes of 0.05 M Acetate, containing 0.5 M NaCl, pH 4.0; and 0.05 M Borate Buffer containing 0.5 M NaCl, pH 8.5. The washes are repeated for at least 3 times with total 15-20 volumes of the buffers. The flow rate of the column washing is approximately 5 cm/min. The gel is equilibrated at 5 cm/min with at least 15-20 volumes of the Wash Buffer, 0.01M Phosphate-Buffered Saline, pH 7.4. The pH of the eluate is checked, and the column is stored at 2-8° C. until use.

### Treatment of HCV-Positive Human Plasma

In some embodiments, Nalcoag silica (Nalco, Naperville, Ill.), 0.2 micron-filtered, is added into the pooled HCV-positive human plasma to give 2.5% (v/v). The mixture is hand-mixed briefly followed by rotation at 15 rpm on a rotator for 4 hours at room temperature. The silica is removed by centrifugation at 4,500 rpm at 2-8° C. in a Beckman RC-3B centrifuge for 30 minutes. The supernatant is filtered through 0.2 micron filter.

Alternatively, the pooled HCV-positive human plasma is fractionated with saturated ammonium sulfate. Saturated ammonium sulfate (SAS) is prepared at room temperature (20-25° C.) in phosphate buffered saline, pH 7.4 using standard laboratory methods. An equal volume of the SAS is gradually added over the course of 10-15 min. to the plasma while stirring. The ammonium sulfate/plasma mixture is stirred at room temperature for 2 hours, and then centrifuged at approximated 4,500 rpm for 30 min at room temperature. The supernatant is decanted, and the pellet is resuspended in phosphate-buffered saline, pH 7.4. The IgG-enriched preparation is filtered though a 0.2 μM filter.

### Solvent and Detergent Treatment of HCV-positive Human Plasma

A 7.5× solvent and detergent concentrate is prepared in phosphate-buffered saline pH 7.4 to give 7.5% Triton X-100 and 2.25% TNBP. The solvent detergent concentrate (7.5×) is slowly added into the fractionated plasma with stirring over approximately 15-20 minutes, to give a final solvent/detergent concentration of 1% Triton X-100 and 0.3% TNBP.

The resultant solvent/detergent/plasma mixture is stirred at room temperature for approximately 4 hrs, and then filtered through a 0.2 μM filter.

The preparation is then subjected to buffer exchange 5 times against the phosphate buffered saline, pH 7.4. The preparation is filter the again preparation through a 0.2 μM filter, to produce an IgG-enriched, solvent/detergent treated preparation that is ready for affinity chromatography.

### IgG Isolation from HCV-positive Human Plasma

In some embodiments, human IgG is isolated from the Solvent/Detergent treated HCV-positive Human Plasma by affinity chromatography over a column packed with ProSep A™ gel (Millipore Corporate, Billerica, Mass.). A Column with packed ProSep A™ gel is sanitized with 120 mM phosphoric acid, 167 mM acetic acid and 2.2% benzyl alcohol or 70% ethanol, pH 3.0 at room temperature for 1 hour, followed by washing with at least 20 volumes of Binding Buffer (1.0 M glycine, 0.15 M NaCl, pH 8.6), at approximately 5 cm/min.

The solvent/detergent treated HCV-positive human plasma is diluted into 2 volumes of the Binding Buffer. ProSep A™ gel's binding capacity is approximately 20 g human IgG per liter. The diluted plasma is charged at approximately 1.25 cm/min over the column, based on the column volume and the gel binding capacity. The volume of the plasma charged can to be adjusted following the aging of the gel.

The column is washed at approximately 5 cm/min with approximately 10 CV of the Binding Buffer to remove the non-IgG fractions.

The bound human IgG is eluted off the column at approximately 5 cm/min with approximately 10 CV of the Elution Buffer, 0.1 M Citrate, pH 3.5. Neutralization Buffer (0.5 M K₂HPO₄, pH 8.5), is added in line to neutralize the IgG fraction to give approximately pH 5.5.

The column is regenerated at approximately 5 cm/min with 10 CV of the Regeneration Buffer, 0.8% Triton X-100 made in de-ionized water adjusted to pH 1.5 with 6 M HCl.

The column is equilibrated at 5 cm/min with 10 CV of the Binding Buffer (1.0 M glycine, 0.15 M NaCl, pH 8.6).

The IgG eluate is concentrated by ultra-filtration with 30 k membranes to give approximately 10-12 mg/ml. The concentrated eluate is filtered through 0.2 μM filter for further purification by affinity chromatography to isolate the pathogen-specific antibodies.

The following example describes the treatment of an individual infected with Hepatitis C virus using the compositions and methods disclosed herein.

### Removal of Antibody Aggregate and Non-IgG Components Column Chromatography

In some embodiments the affinity-purified, pathogen-specific human antibodies are further processed by ion exchange chromatography over a Ceramic Hydroxyapatide (CHT) column (approximately 20 cm in bed height) which is equilibrated with CHT Wash Buffer, 0.05 M glycine, pH 5.5. The affinity-purified antibodies are buffer-exchanged 5 times against CTH Wash Buffer and concentrated to 8-12 mg/ml. The buffer-exchanged antibody preparation is loaded over the CHT, followed by isocratic wash with CHT Wash Buffer until A₂₈₀ drops to below the baseline (<0.1 unit). The monomeric IgG is disassociated from the CHT functional groups by a step gradient with Elution Buffer, 0.35 M NaCl made in CHT Wash Buffer, pH 5.5. IgG fractions are collected and tested for monomeric IgG by HPLC gel filtration chromatography. The fractions with IgG monomers and dimmers greater than 90% are pooled for buffer exchanged into 10% Maltose/0.03% Polysorbate 80, pH 5.5. The product is 0.2 μM filtered and tested before its intended application in patient treatment.

As an alternative to ion exchange (CHT) chromatography, in some embodiments, the affinity-purified, pathogen-specific human antibodies are further processed by other chromatographic methods, including but not limited to gel filtration, e.g., Sepharcyl-S-200, Sephacryl-S-300 (GE Lifesciences), or the like; ion exchange chromatography with a CAPTO™ adhere (GE Lifesciences), Poros HQ (Applied Biosystems, Inc.), or DEAE (GE Lifesciences) column, or the like; or hydrophobic interaction chromatography, e.g., using a Buty-S-Sepharose 6 Fast Flow Lifesciences) chromatography.

### Example 2

A subject is identified as being infected with Hepatitis C virus using routine diagnostic methods. The subject is administered a dose of the composition described in Example 1 by intravenous injection. Preferably, the dosage of anti-HCV antibody in the injection is in the range from about 100-200 mg of affinity purified human anti HCV.

The titer of HCV (or viral load) present in the subject's blood is determined before and after administration of the anti-HCV polyclonal antibody cocktail from Example 1. Depending on the viral load, the subject can be administered one or more additional doses. At the discretion of the medical care provider, the individual can receive additional administrations, e.g., the individual receives a single dose every 2 weeks for 60 weeks.

The following example describes the treatment of an individual with HIV using the compositions and methods disclosed herein.

### Example 3

A subject is identified as being infected with Human Immunodeficiency Virus (HIV) using routine diagnostic methods. The subject is administered a dose of a composition that includes a mixture of anti-HIV polyclonal antibodies that have been affinity purified from the plasma of several HIV positive individuals. The composition is parenterally administered. Preferably, the dosage of anti-HCV antibody in the composition is in the range from about 100-200 mg.

The titer of HIV (or viral load) present in the subject's blood is determined before and after administration of the anti-HIV polyclonal antibody cocktail. Depending on the viral load, the subject can be administered one or more additional doses.

The following example describes the treatment of an individual with L. monocytogenes using the compositions and methods disclosed herein.

### Example 4

A subject is identified as being infected with a L. monocytogenes using routine diagnostic methods. The subject is administered a dose of a composition that includes a mixture of anti-L. monocytogenes polyclonal antibodies that have been affinity purified from the plasma of several individuals. The composition is intravenously administered. Preferably, the dosage of anti-L. monocytogenes antibody in the composition is in the range from about 100-200 mg.

The presence of L. monocytogenes in the subject's blood is determined before and after administration of the anti-L. monocytogenes polyclonal antibody cocktail. The subject can be tested for the presence of L. monocytogenes following administration of the composition. Depending on whether L. monocytogenes is detectable following the first dose of the polyclonal antibodies, the subject can be administered one or more additional doses.

The following example demonstrates that normal human serum contains anti-H1N5 (avian influenza virus) antibodies.

### Example 5

### Plasma Source

3.5 L of normal human serum was lipid stripped as described in Example 1. The lipid-stripped serum was applied without solvent/detergent treatment.

### Affinity Column Preparation

A peptide derived from H1N5 influenza virus (LSLRNPILV)(SEQ ID NO:11) was synthesized using standard techniques (GENESCRIPT™, Piscataway, N.J.). SEQ ID NO: 11 is derived from a conserved protein of 87 amino acids, PB1-F2. PB1-F2 has unusual features relative to other influenza virus gene products including variable expression in individual infected cells, rapid proteasome-dependent degradation, and mitochondrial localization.

5 mg f the polypeptide was reconstituted in 0.5 mL distilled water and diluted into approximately 3 mL 0.05M Borate Buffer, containing 0.5M NaCl, pH 8.5.

2 g CNBr-activated Sepharose 4B dry powder (GE Healthcare Bio-Sciences™, Piscataway, N.J.), was suspended in 0.001N HCl, pH 3. The gel slurry was packed into a glass column housing and washed with 0.001N HCL, pH 3, followed by brief wash with 0.05M Borate Buffer, containing 0.5M NaCl, pH 8.5. The gel (approximately 7 mL) was drained and the peptide solution was loaded onto the column, followed by manually mixing, placing the column on a rotator at approximately 15 rpm for 1 hr. at room temperature, followed by overnight at 2-8° C.

The column was drained, and the remaining active sties were blocked with 2 column volumes of 1.0M glycine made in 0.05M Borate Buffer, containing 0.5M NaCl, pH 8.5 at 2-8° C. for 1 hour.

The gel was washed with 3.5 volumes of 0.05 M Acetate, containing 0.5 M NaCl, pH 4.0; and 3.5 volumes of 0.05 M Borate Buffer, containing 0.5 M NaCl, pH 8.5. The washes were performed at a rate of 5 cm/min, and repeated twice.

The column was equilibrated with at least 20 volumes of the Wash Buffer, 0.01M Phosphate-Buffered Saline, pH 7. The equilibration was performed at a rate of 4. 5 cm/min.

### IgG Isolation from Normal Human Plasma

The column was charged with 3.5L of lipid-stripped human serum, at approximately 1.1 mL/min, over 52 hours at 2-8° C. The column was washed with Phosphate Buffered Saline, pH 7.4, at 7.5 ml/min for 45 minutes.

Antibodies that specifically bound the peptide were eluted from the column with 0.1 M Glycine, pH 2.75, at approximately 5 ml/min. into a Conical tube containing 2.5 ml of 0.5 M Phosphate Buffer, pH 8.5. The pH of the eluate was adjusted to pH 7.4.

Using this procedure, 16 ml anti-PB1-F2 (62-70) polyclonal antibody was collected, amounting to 11.8 mg, as measured at A₂₈₀. (OD of 1.033)

### Gel Filtration Analysis of Eluate

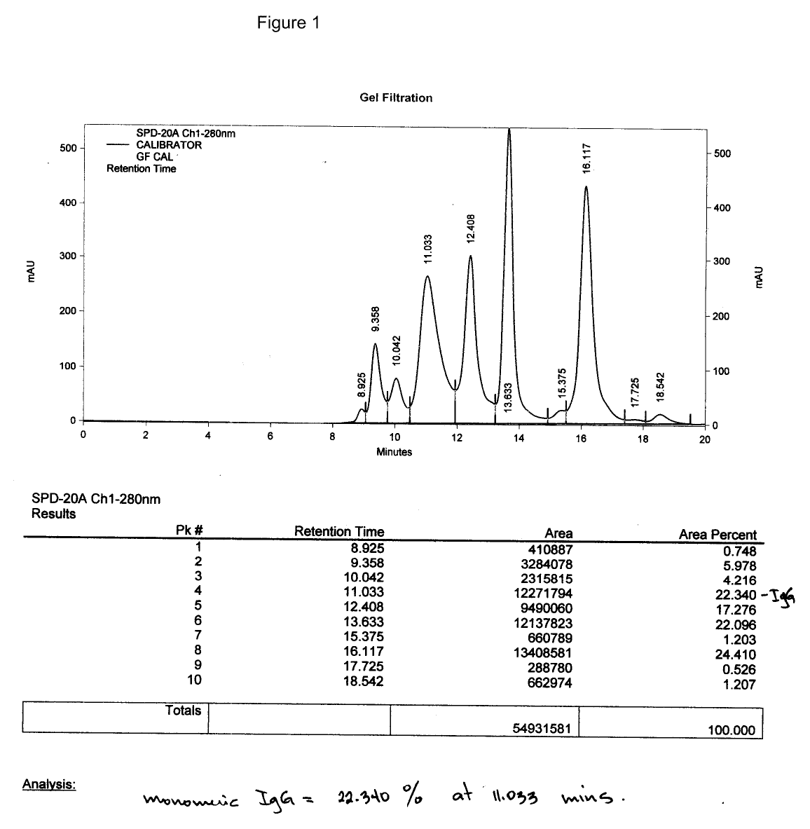

A gel filtration column was calibrated with the following standards: thyroglobulin (670 KDa), bovine gamma globulin (158 KDa), chicken ovalbumin (44 KDa), equine myoglobin (17 KDa) and vitamin B12 (1.35 KDa). The retention time for each of the standards was measured and is reflected in FIG. 1. The bovine gamma globulin standard eluted at 11.033 minutes.

The antibody eluate from PB1-F2 affinity purification step described above was run over the gel filtration column using the same conditions. The retention time for the peak was 10.958 minutes, which overlapped with the bovine gamma globulin standard. Further, the results showed that the purified product contained 70.4% monomeric IgG.

### ELISA Analysis of Eluate

In order to test the specificity of the purified antibodies for the PB1-F2 antigen, an ELISA assay was performed. 1 μg purified PB1-F2 (62-70) was covalently linked to the wells of six rows of a 96-well microtiter plate using routine techniques. As a control, 1 μg purified human PTH antigen (7-84) was coupled to the remaining wells of the microtiter plate. The plate was blocked for 2 hours with 1% BSA and air-dried overnight.

100 μL of the purified human antibody (the eluate) described above, was added to each well and incubated at room temperature for three hours. The plate was rocked at 350 rpm.

The plate was washed 5× with 350 μL standard ELISA wash buffer. Anti-human IgG-HRP (SIGMA®, St. Louis, Mo.) was diluted into 1% BSA. 100 μL of the secondary antibody was added to each well. The secondary antibody was incubated at room temperature for one hour. The ELISA plate was washed 5× with standard wash buffer. 150 μL 3,3,′5,5′-tetramethylbenzidine (TMB) was added to each well. The TMB substrate was incubated at room temperature for 30 minutes. 100 μL standard ELISA stop solution was added to each well. The OD₄₅₀ for each of the wells was determined, and the difference in OD₄₅₀ (delta OD₄₅₀) between the negative control wells and the PB1-F2 coupled wells was determined. The data are provided in Table 3, below.

TABLE 3

| | Antibody added | | | | |
|---|---|---|---|---|---|
| | 7.4 μg | 0.74 μg | 0.3 μg | 0.15 μg | 0.075 μg |
| Delta OD₄₅₀ | 0.253 | 0.037 | 0.014 | 0.004 | 0.015 |

The data demonstrate that normal human plasma contains antibodies that are specific to H1N5 antigens.

### Example 6

Mice are divided into two groups. All mice are infected with live H1N1 virus by nasal spray.

10 minutes after infection with H1N1, the mice in the experimental group are injected with the composition purified over a column with immobilized H1N1 peptides, described herein above. At the same time, mice in the control group are injected with an equal volume of phosphate buffered saline.

The mice in the control group die within six days. The mice in the experimental group do not die, demonstrating that the antibodies from normal human plasma are useful for treatment of individuals infected with H1N1.

### Example 7

A subject is identified as being exposed to H1N1. The subject is administered a dose of a composition that includes a mixture of anti-H1N1 polyclonal antibodies that have been affinity purified from the plasma of several individuals. The composition is intravenously administered. Preferably, the dosage of anti-H1N1 antibody in the composition is in the range from about 100-200 mg. The subject receives multiple administrations of the H1N1 polyclonal antibodies at regular intervals.

The presence of H1N1 in the subject's blood is determined before and after administration of the anti H1N1 polyclonal antibody cocktail. The subject is tested for the presence of H1N1 following administration of the composition. Depending on whether H1N1 is detectable following the first dose of the polyclonal antibodies, the subject can be administered one or more additional doses.

What is claimed is:

1. A composition comprising a mixture of polyclonal antibodies obtained from a plurality of individual subjects in combination with a pharmacologically-acceptable carrier, wherein said mixture of polyclonal antibodies comprises antibodies that specifically bind to at least one antigen of a target pathogen, and wherein said polyclonal antibodies or said mixture has been processed to substantially separate antibodies that do not specifically bind to said at least one antigen of said target pathogen from said polyclonal antibodies.

2. (canceled)

3. (canceled)

4. (canceled)

5. The composition of claim 1, wherein said at least one antigen is derived from at least one peptide found in Hepatitis C (HCV).

6. The composition of claim 4, wherein said at least one antigen is derived from at least one recombinant protein that contains at least one Hepatitis C epitope.

7. (canceled)

8. The composition of claim 7, wherein said target is an H1N1 viral particle.

9. (canceled)

10. (canceled)

11. The composition of claim 1, wherein said plurality of individual subjects are collectively infected with a plurality of HCV strains.

12. The composition of claim 8, wherein said plurality of individual subjects are collectively infected with a plurality of H1N1 strains.

13. The composition of claim 1, wherein the amount of polyclonal antibodies in said composition comprises between about 0.1 to about 10mg polyclonal antibodies per milliliter.

14. (canceled)

15. (canceled)

16. A method of preparing the composition of claim 1, comprising:
providing plasma from a plurality of individual subjects;
combining said plasma from said plurality of subjects to obtain a plasma mixture;
contacting said plasma mixture with the at least one immobilized antigen of said pathogenic target or related pathogenic target under conditions wherein said polyclonal antibodies within said plasma that specifically bind to said at least one antigen bind to said at least one antigen;
separating said polyclonal antibodies from antibodies (and other non specific proteins) that do not specifically bind said at least one antigen of said pathogenic target; and
providing the separated antibodies in combination with a pharmacologically-acceptable carrier.

17. (canceled)

18. The method of claim 16, further comprising isolating the gamma globulin component from said plasma mixture prior to said contacting step, and wherein said gamma globulin component is contacted with the at least one antigen from said pathogenic target or related pathogenic target.

19. The method of claim 16, further comprising:
exposing said bound polyclonal antibodies to conditions wherein said polyclonal antibodies dissociate from said at least one immobilized antigen from said pathogenic target following said separating step; and
separating said dissociated polyclonal antibodies from said at least one antigen.

20. The method of claim 19, further comprising:
concentrating said separated, dissociated polyclonal antibodies.

21. The method of claim 19, further comprising
purifying monomeric forms of said polyclonal antibodies from non monomeric forms of said polyclonal antibodies.

22. (canceled)

23. The method of claim 16, wherein said pathogenic target is a Hepatitis C (HCV) viral particle.

24. (canceled)

25. The method of claim 16, wherein said pathogenic target is an H1N1 viral particle.

26. The method of claim 16, wherein the amount of polyclonal antibodies in said composition is between about 0.1 mg and 20 mg per milliliter.

27. (canceled)

28. (canceled)

29. The method of claim 23, wherein said plurality of individual subjects are collectively infected with a plurality of HCV strains.

30. The method of claim 25, wherein said plurality of individual subjects are collectively infected with a plurality of H1N1 strains.

31. A method of treating a disease caused by a pathogenic target, wherein said pathogenic target is selected from the group consisting of a viral particle, a pathogenic microorganism, a pathogenic fungus, and a pathogenic parasite, comprising:
identifying a first subject that is infected with said pathogenic target; and
administering to said subject a therapeutically effective amount of a composition comprising a mixture of polyclonal antibodies obtained from a plurality of individual subjects other than said first subject, wherein said polyclonal antibodies specifically bind to said pathogenic target and wherein said polyclonal antibodies have been processed to separate antibodies that do not specifically bind to said pathogenic target from said polyclonal antibodies.

32. (canceled)

33. The method of claim 3231, wherein said target is a Hepatitis C (HCV) viral particle.

34. (canceled)

35. The method of claim 31, wherein said target is an H1N1 viral particle.

36. (canceled)

37. (canceled)

38. The method of claim 31, wherein said composition is administered intravenously.

39. The method of claim 31, wherein said composition is administered into the nose of the subject.

40. (canceled)

41. (canceled)

42. The method of claim 33, wherein said plurality of individual subjects are collectively infected with a plurality of HCV strains.

43. The method of claim 31, wherein said plurality of individual subjects comprises individuals that are infected with said pathogenic target.

44. (canceled)

45. (canceled)

46. The composition of claim 1, wherein said plurality of individual subjects comprises individuals that are not infected with said pathogenic target.

47. (canceled)

48. The method of claim 16, wherein said plurality of individual subjects comprises individuals that are not infected with said pathogenic target.

49. (canceled)

50. The method of claim 31, wherein said plurality of individual subjects comprises individuals that are not infected with said pathogenic target.

51. (canceled)

52. (canceled)

53. (canceled)

54. (canceled)

55. (canceled)

56. (canceled)

57. The method of claim 16, further comprising removing endogenous lipids from said plasma or plasma mixture.

58. (canceled)

59. (canceled)

60. (canceled)

61. (Cancelled)

62. A composition comprising a mixture of a plurality of monoclonal antibodies, wherein each monoclonal antibody of said plurality specifically binds an epitope on a target pathogen.
